(19) United States
(12) Patent Application Publication
(10) Pub. No.: US 2020/0384614 A1
(43) Pub. Date: Dec. 10, 2020

(54) Multi-Pronged Tweezer

(71) Applicants: Muhammad Hussain, Highland Park, NJ (US); Farah Hussain, Highland Park, NJ (US)

(72) Inventors: Muhammad Hussain, Highland Park, NJ (US); Farah Hussain, Highland Park, NJ (US)

(21) Appl. No.: 16/728,404

(22) Filed: Dec. 27, 2019

Related U.S. Application Data
(63) Continuation-in-part of application No. 29/694,396, filed on Jun. 10, 2019.

(51) Int. Cl. B25B 9/02; A61B 17/30; A45D 26/00

(52) U.S. Cl. CPC B25B 9/02; A61B 2017/303; A45D 26/0066; A61B 17/30

(57) ABSTRACT

A multi-pronged tweezer is an apparatus that provides a variety of tips in order to effectively remove or position small items. The apparatus includes an elongated tweezing body, a first tip pair, a second tip pair, a third tip pair. The elongated tweezing body further includes a first tweezing arm, a second tweezing arm, a stabilizing tweezing arm, first tweezing end, and a second tweezing end. The first tweezing arm and the second tweezing arm position and orient the first tip pair, the second tip pair, and the third tip pair. The first tweezing arm and the second tweezing arm serve function as levers as the stabilizing tweezing arm transversally connects in between the first tweezing arm and the second tweezing arm. The first tip pair is slanted tip pair, the second tip pair is a straight tip pair, and the third tip pair is a pointed tip pair.

The current application is a continuation-in-part (CIP) application of the U.S. design application Ser. No. 29/694,396 filed on Jun. 10, 2019.

### FIELD OF THE INVENTION

The present invention generally relates to tweezers. More specifically, the present invention is a multi-pronged tweezer.

### BACKGROUND OF THE INVENTION

A variety of cosmetic tools are required to accomplish specific tasks. In order to properly apply makeup, various types of brushes are needed to apply specific types of makeup. Specific accessories and processes enhance the effectiveness of skincare products. A variety tools are also necessary for grooming. Specific tools are necessary for each grooming task. One common grooming task is hair removal. Hair removal may be as simple as eyebrow grooming or as tedious as ingrown hair removal. Another grooming task that requires delicate application is false eyelash application.

With a variety of tweezer ends better suited for each task, it is an objective of the present invention to provide a universal tweezer that may tend to a variety of skincare, makeup, and grooming needs. The present invention is a multi-pronged tweezer with slanted tip, a straight tip, and a third tip. The arrangement of each tip with each other allows each tip to securely grasp a small item with minimal effort. The present invention also allows each tip to be used independently of one another without a user getting scratched or the skin being damaged by one of the tips not being used.

It is understood that the present invention may also be used for a variety of activities that involve the manipulation and positioning of small items. The present invention may be used in vary arts and crafts and do-it-yourself projects. The present invention may also be used in medical procedures and tasks such as those seen in dental care or dermatology services.

### BRIEF DESCRIPTION OF THE DRAWINGS

FIG. 1 is a front perspective view of the present invention.

FIG. 2 is a rear perspective view of the present invention.

FIG. 3 is a top-side view of the present invention.

FIG. 4 is a right-side view of the present invention.

FIG. 5 is a left-side view of the present invention.

### DETAIL DESCRIPTIONS OF THE INVENTION

All illustrations of the drawings are for the purpose of describing selected versions of the present invention and are not intended to limit the scope of the present invention.

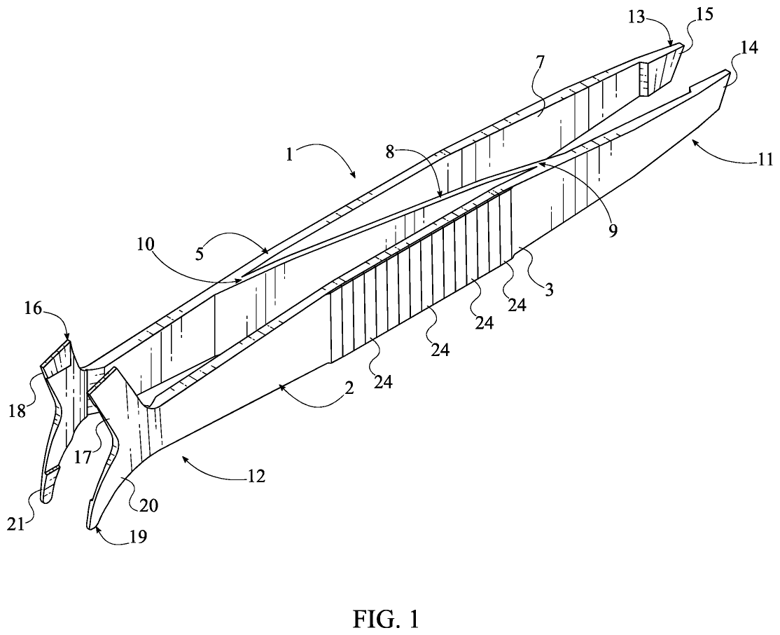

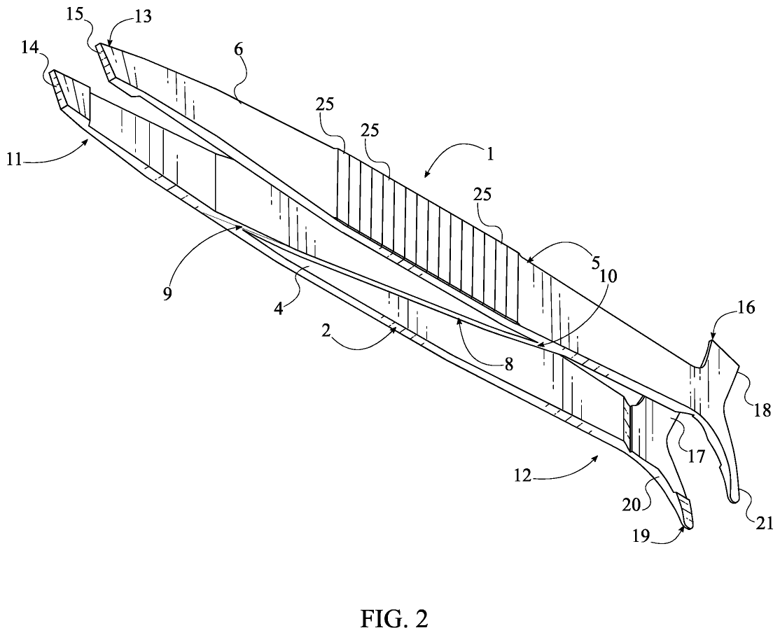

The present invention facilitates tweezing for a user for a variety of small items that require precision and are too small to be picked up with fingers. The present invention is able to manage the removal ingrown hairs, small body hairs, and a variety of other extractions that cannot be removed with fingers of a user. The present invention also allows a user to position small fine objects onto the skin of a user or a surface. The present invention serves as a universal tool for the removal of or placement of small items as the present invention comprises an elongated tweezing body 1, a first tip pair 13, a second tip pair 16, and a third tip pair 19, seen in FIG. 1 and FIG. 2. The elongated tweezing body 1 positions and orients the first tip pair 13, the second tip pair 16, and the third tip pair 19 with each other. The elongated tweezing body 1 preferably comprises metal materials. However, it is understood that various embodiments of the present invention may comprise a variety of rigid materials. Moreover, the first tip pair 13, the second tip pair 16, and the third tip pair 19 are able to each serve as a lever as the elongated tweezing body 1 comprises a first tweezing arm 2, a second tweezing arm 5, a stabilizing tweezing arm 8, a first tweezing end 11, and a second tweezing end 12. A user is able to maneuver the first tip pair 13, the second tip pair 16, and the third tip pair 19 with the first tweezing arm 2 and the second tweezing arm 5. The stabilizing tweezing arm 8 allows a user to maneuver small objects with the first tweezing arm 2 and the second tweezing arm 5. The first tweezing end 11 orients the first tip pair 13, and the second tweezing end 12 orients both the second tip pair 16 and the third tip pair 19. The first tip pair 13, the second tip pair 16, and the third tip pair 19 directly grasp onto small objects, each of which are contoured to grasp onto specific small objects.

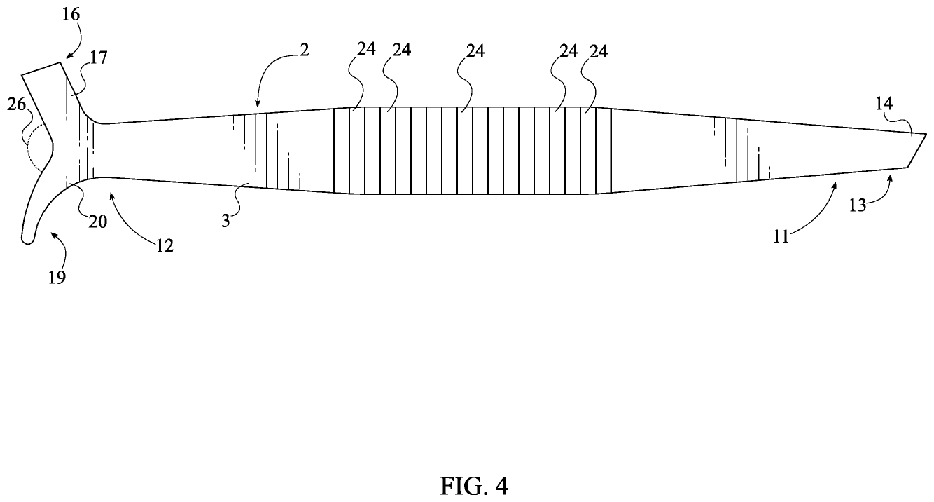

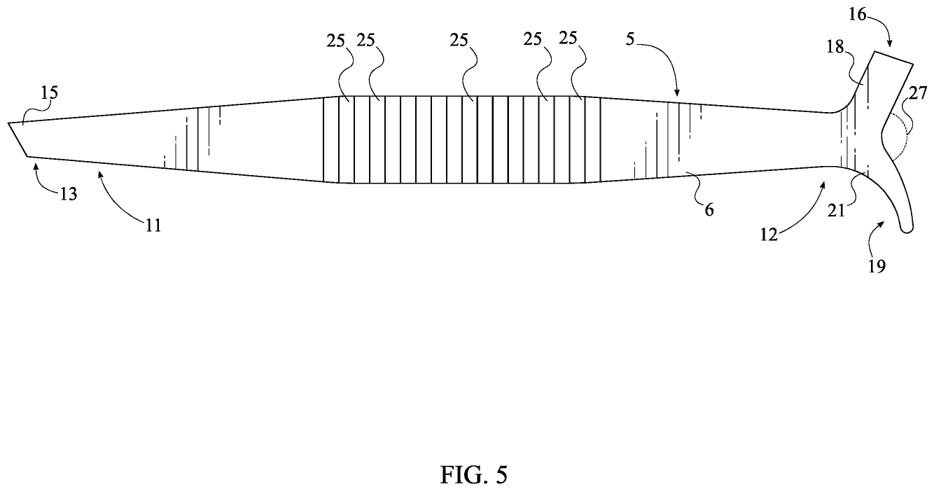

In the preferred embodiment of the present invention, seen in FIG. 4 and FIG. 5, the first tip pair 13 is a slanted tip pair. The slanted tip pair allows the first tip pair 13 to be positioned near the base of the small object against an adjacent surface such as skin. The slanted base is typically good for picking up fine objects such as a strand of hair from a layer of skin. The second tip pair 16 is preferably a straight tip pair. The straight tip pair allows small items to be positioned and delicately placed onto a surface such as skin. The straight tip pair is preferably used to apply fine items such as eyelashes. The straight tip pair may also be referred to as a flat square tip pair. The third tip pair 19 is a pointed tip pair that facilitates the remove of items that may not fully traverse through the surface. The pointed tip allows a user to tackle ingrown hairs and further contour facial hair. The pointed tip provides enhanced precision for more difficult areas.

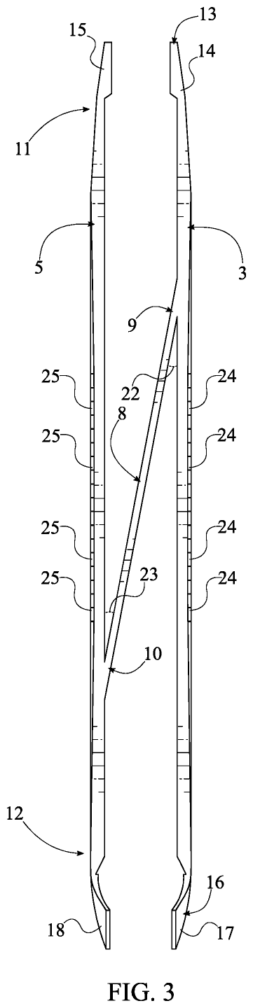

The overall configuration of the aforementioned components allows a user to firmly grasp onto a variety of small items with minimal effort. Small items are positioned between either the first tip pair 13, the second tip pair 16, and the third tip pair 19 as the first tweezing arm 2 is positioned parallel and offset from the second tweezing arm 5, seen in FIG. 3. The first tweezing end 11 is positioned opposite the second tweezing end 12 along the elongated tweezing body 1 so that the first tip pair 13 is offset from both the second tip pair 16 and third tip pair 19. The first tweezing arm 2 and the second tweezing arm 5 each function as a lever with the stabilizing tweezing arm 8 as the stabilizing tweezing arm 8 is transversally connected in between the first tweezing arm 2 and the second tweezing arm 5. More specifically, first rod end 9 of the stabilizing tweezing arm 8 is positioned adjacent with the first tweezing end 11. Similarly, a second rod end 10 of the stabilizing tweezing arm 8 is positioned adjacent with the second tweezing end 12. In order for a small object to be grasped with the first tip pair 13, a first tip 14 of the first tip pair 13 is terminally fixed with the first tweezing arm 2, adjacent with the first tweezing end 11, and a second tip 15 of the first tip pair 13 is terminally fixed with the second tweezing arm 5, adjacent with the first tweezing end 11. Similarly, a first tip 17 of the second tip pair 16 and a first tip 20 of the third tip pair 19 are terminally fixed with the first tweezing arm 2, adjacent with the second tweezing end 12. Moreover, a second tip 18 of the second tip pair 16 and a second tip 21 of the third tip pair 19 are terminally fixed with the second tweezing arm 5, adjacent with the second tweezing end 12. The second tip pair 16 and the third tip pair 19 engage independently with a small object as the first tip 17 of the second tip pair 16 and the first tip 20 of the third tip pair 19 are oriented away from each other. Likewise, the second tip 18 of the second tip pair 16 and the second tip 21 of the third tip pair 19 are oriented away from each other.

The elongated tweezing body 1 requires minimal effort to squeeze the first tweezing arm 2 and the second tweezing arm 5 with each other as the stabilizing arm is oriented at a first acute angle 22 with the first tweezing arm 2 and oriented at a second acute angle 23 with the second tweezing arm 5, also seen in FIG. 3. More specifically, the first acute angle 22 and the second acute angle 23 are alternate interior angles between the first tweezing arm 2 and the second tweezing arm 5. This arrangement preserves the parallel orientation between the first tweezing arm 2 and the second tweezing arm 5. Furthermore, this allows the small objects to be firmly grasped with either the first tip pair 13, the second tip pair 16, or third tip pair 19. The first tip 14 and the second tip 15 for the first tip pair 13, the second tip pair 16, and the third tip pair 19, respectively, are aligned with each other as the first acute angle 22 and the second acute angle 23 are equal to each other.

As seen in FIG. 1, FIG. 2, FIG. 4, and FIG. 5, the present invention further comprises a plurality of first ridges 24 and a plurality of second ridges 25 in order to secure the finger placement of the user along the first tweezing arm 2 and the second tweezing arm 5, respectively, throughout the engagement of the elongated tweezing body 1. The plurality of first ridges 24 and the plurality of second ridges 25 may comprise a variety of designs in order to reflect the stylistic preferences of the user. The finger placement of the user is secure along the first tweezing arm 2 as the first tweezing arm 2 comprises a first surface 3 and a second surface 4, and the first surface 3 is positioned opposite the second surface 4 about the first tweezing arm 2. This arrangement is seen in FIG. 1 and FIG. 2. More specifically, the first surface 3 and the second surface 4 are positioned parallel with each other. The stabilizing tweezing arm 8 is positioned adjacent with the second surface 4, and the plurality of first ridges 24 are integrated into the first surface 3. This arrangement allows the fingers of a user to press on the plurality of first ridges 24 while squeezing the elongated tweezing body 1. Moreover, the plurality of first ridges 24 is distributed across the first surface 3.

The finger placement of the user is secure along the second tweezing arm 5 as the second tweezing arm 5 also comprises a first surface 6 and a second surface 7, and the first surface 6 is positioned opposite the second surface 7 about the second tweezing arm 5. This arrangement is seen in FIG. 1 and FIG. 2. More specifically, the first surface 6 and the second surface 7 are positioned parallel with each other. Similar with the first tweezing arm 2, the stabilizing tweezing arm 8 is positioned adjacent with the second surface 7, and the plurality of second ridges 25 is integrated into the first surface 6. Moreover, the plurality of second ridges 25 is distributed across the first surface 6.

The present invention preserves an ergonomic structure as the first tip 17 of the second tip pair 16 and the first tip 20 of the third tip pair 19 are oriented at a first obtuse angle 26, seen in FIG. 4 and FIG. 5. The first tip and the second tip for both the second tip pair 16 and the third tip pair 19 are aligned with each other respectively, as the second tip 18 of the second tip pair 16 and the second tip 21 of the third tip pair 19 are oriented at a second obtuse angle 27 as well. The first obtuse angle 26 and the second obtuse angle 27 allow the second tip pair 16 and the third tip pair 19 to be engaged independently of one another without the user getting scratched or the surface getting scratched. In the preferred embodiment of the present invention, the first obtuse angle 26 and the second obtuse angle 27 are equal with each other.

Although the invention has been explained in relation to its preferred embodiment, it is to be understood that many other possible modifications and variations can be made without departing from the spirit and scope of the invention as hereinafter claimed.

What is claimed is:

1. A multi-pronged tweezer comprises:
an elongated tweezing body;
a first tip pair;
a second tip pair;
a third tip pair;
the elongated tweezing body comprises a first tweezing arm, a second tweezing arm, a stabilizing tweezing arm, a first tweezing end, and a second tweezing end;
the first tweezing arm being positioned parallel and offset from the second tweezing arm;
the first tweezing end being positioned opposite the second tweezing end along the elongated tweezing body;
the stabilizing tweezing arm being transversally connected in between the first tweezing arm and the second tweezing arm;
a first rod end of the stabilizing tweezing arm being positioned adjacent with the first tweezing end;
a second rod end of the stabilizing tweezing arm being positioned adjacent with the second tweezing end;
a first tip of the first tip pair being terminally fixed with the first tweezing arm, adjacent with the first tweezing end;
a second tip of the first tip pair being terminally fixed with the second tweezing arm, adjacent with the first tweezing end;
a first tip of the second tip pair and a first tip of the third tip pair being terminally fixed with the first tweezing arm, adjacent with the second tweezing end;
a second tip of the second tip pair and a second tip of the third tip pair being terminally fixed with the second tweezing arm, adjacent with the second tweezing end;
the first tip of the second tip pair and the first tip of the third tip pair being oriented away from each other; and,
the second tip of the second tip pair and the second tip of the third tip pair being oriented away from each other.

2. The multi-pronged tweezer as claimed in claim 1 comprises:
the stabilizing arm being oriented at a first acute angle with the first tweezing arm;
the stabilizing arm being oriented at a second acute angle with the second tweezing arm; and,
the first acute angle and the second acute angle being alternate interior angles in between the first tweezing arm and the second tweezing arm.

3. The multi-pronged tweezer as claimed in claim 2 comprises:
the first acute angle and the second acute angle being equal to each other.

4. The multi-pronged tweezer as claimed in claim 1 comprises:
a plurality of first ridges;
the first tweezing arm comprises a first surface and a second surface;
the first surface being positioned opposite the second surface about the first tweezing arm;
the first surface and the second surface being positioned parallel with each other;
the stabilizing tweezing arm being positioned adjacent the second surface;
the plurality of first ridges being integrated into the first surface; and,
the plurality of first ridges being distributed across the first surface.

5. The multi-pronged tweezer as claimed in claim 1 comprises:
a plurality of second ridges;
the second tweezing arm comprises a first surface and a second surface;
the first surface being positioned opposite the second surface about the second tweezing arm;
the first surface and the second surface being positioned parallel with each other;
the stabilizing tweezing arm being positioned adjacent the second surface;
the plurality of second ridges being integrated into the first surface; and,
the plurality of second ridges being distributed across the first surface.

6. The multi-pronged tweezer as claimed in claim 1 comprises:
the first tip of the second tip pair and the first tip of the third tip pair being oriented at a first obtuse angle;
the second tip of the second tip pair and the second tip of the third tip pair being oriented at a second obtuse angle; and,
the first obtuse angle and the second obtuse angle being equal to each other.

7. The multi-pronged tweezer as claimed in claim 1 comprises:
the first tip pair being a slanted tip pair.

8. The multi-pronged tweezer as claimed in claim 1 comprises:
the second tip pair being a straight tip pair.

9. The multi-pronged tweezer as claimed in claim 1 comprises:
the third tip pair being a pointed tip pair.

10. A multi-pronged tweezer comprises:
an elongated tweezing body;
a first tip pair;
a second tip pair;
a third tip pair;
the elongated tweezing body comprises a first tweezing arm, a second tweezing arm, a stabilizing tweezing arm, a first tweezing end, and a second tweezing end;
the first tweezing arm being positioned parallel and offset from the second tweezing arm;
the first tweezing end being positioned opposite the second tweezing end along the elongated tweezing body;
the stabilizing tweezing arm being transversally connected in between the first tweezing arm and the second tweezing arm;
a first rod end of the stabilizing tweezing arm being positioned adjacent with the first tweezing end;
a second rod end of the stabilizing tweezing arm being positioned adjacent with the second tweezing end;
a first tip of the first tip pair being terminally fixed with the first tweezing arm, adjacent with the first tweezing end;
a second tip of the first tip pair being terminally fixed with the second tweezing arm, adjacent with the first tweezing end;
a first tip of the second tip pair and a first tip of the third tip pair being terminally fixed with the first tweezing arm, adjacent with the second tweezing end;
a second tip of the second tip pair and a second tip of the third tip pair being terminally fixed with the second tweezing arm, adjacent with the second tweezing end;
the first tip of the second tip pair and the first tip of the third tip pair being oriented away from each other;
the second tip of the second tip pair and the second tip of the third tip pair being oriented away from each other;
the stabilizing arm being oriented at a first acute angle with the first tweezing arm;
the stabilizing arm being oriented at a second acute angle with the second tweezing arm; and,
the first acute angle and the second acute angle being alternate interior angles in between the first tweezing arm and the second tweezing arm.

11. The multi-pronged tweezer as claimed in claim 10 comprises:
the first acute angle and the second acute angle being equal to each other.

12. The multi-pronged tweezer as claimed in claim 10 comprises:
a plurality of first ridges;
the first tweezing arm comprises a first surface and a second surface;
the first surface being positioned opposite the second surface about the first tweezing arm;
the first surface and the second surface being positioned parallel with each other;
the stabilizing tweezing arm being positioned adjacent the second surface;
the plurality of first ridges being integrated into the first surface; and,
the plurality of first ridges being distributed across the first surface.

13. The multi-pronged tweezer as claimed in claim 10 comprises:
a plurality of second ridges;
the second tweezing arm comprises a first surface and a second surface;
the first surface being positioned opposite the second surface about the second tweezing arm;
the first surface and the second surface being positioned parallel with each other;
the stabilizing tweezing arm being positioned adjacent the second surface;
the plurality of second ridges being integrated into the first surface; and,
the plurality of second ridges being distributed across the first surface.

14. The multi-pronged tweezer as claimed in claim 10 comprises:
the first tip of the second tip pair and the first tip of the third tip pair being oriented at a first obtuse angle;
the second tip of the second tip pair and the second tip of the third tip pair being oriented at a second obtuse angle; and,
the first obtuse angle and the second obtuse angle being equal to each other.

15. The multi-pronged tweezer as claimed in claim 10 comprises:
the first tip pair being a slanted tip pair.

16. The multi-pronged tweezer as claimed in claim 10 comprises:
the second tip pair being a straight tip pair.

17. The multi-pronged tweezer as claimed in claim 10 comprises:
the third tip pair being a pointed tip pair.
